(19) United States
(12) Patent Application Publication
(10) Pub. No.: US 2018/0028497 A1
(43) Pub. Date: Feb. 1, 2018

(54) PANOBINOSTAT DOSAGES FOR MULTIPLE MYELOMA

(71) Applicant: Novartis AG, Basel (CH)

(72) Inventors: Rong Lin, Martinsville, NJ (US); Renaud Capdeville, Riedesheim (FR); Laura Grazioli, Basel (CH); Song Mu, Basking Ridge, NJ (US); Sofia Paul, Warren, NJ (US); Florence Binlich, Ville D'Avray (FR)

(73) Assignee: Novartis AG, Basel (CH)

(21) Appl. No.: 15/551,768

(22) PCT Filed: Feb. 17, 2016
(86) PCT No.: PCT/IB2016/005085

§ 371 Date: Aug. 17, 2017

Related U.S. Application Data
(60) Provisional application No. 62/119,571, filed on Feb. 23, 2015.
(60) Provisional application No. 62/118,254, filed on Feb. 19, 2015.

(51) Int. Cl. A61K 31/4045; A61K 31/573; A61K 9/00; A61K 31/69

(52) U.S. Cl. CPC A61K 31/4045; A61K 31/69; A61K 31/573; A61K 9/0019

(57) ABSTRACT

Treatment of multiple myeloma with a combination of panobinostat and bortezomib at specified doses adjusted for safety.

### FIELD OF THE DISCLOSURE

The present disclosure relates to administration of a combination of panobinostat and bortezomib at dosages that enhance patient safety. The disclosure further relates to a medicament of panobinostat and bortezomib at dosages that enhance patient safety.

### BACKGROUND OF THE INVENTION

Panobinostat is a pan histone deacetylase (HDAC) inhibitor that works by blocking key cell enzymes implicated in cancer which ultimately leads to cellular stress and death of these cells. Development history and the pharmacological profile of panobinostat and its potential for treatment are described in P. Atadja, Development of the pan-DAC inhibitor panobinostat (LBH589): Successes and challenges, Cancer Letters 280 (2009), 233-241 and in M. Anne et al., Profile of panobinostat and its potential for treatment in solid tumors: an update, OncoTargets and Therapy 2013:6 1613-1624.

In Phase III clinical trials, panobinostat showed significant clinical benefit to patients with multiple myeloma, a cancer that affects approximately 1 to 5 in every 100,000 people worldwide each year. There are currently no curative therapies available for multiple myeloma. Moreover, almost all patients with multiple myeloma ultimately relapse and become resistant to treatment. Therefore, there is a high unmet medical need for therapies addressing this medical condition. There is also a desire to treat multiple myeloma with drugs that have different mechanisms of action. There are currently no HDAC inhibitor drugs that are approved to treat multiple myeloma, which also creates an unmet medical need.

Panobinostat has been subject to ongoing extensive clinical trials by Applicant. The PANORAMA-1 clinical study (PANobinostat ORA1 in Multiple MyelomA) showed that adding panobinostat to a combination of bortezomib and dexamethasone in patients with relapsed or relapsed and refractory multiple myeloma offers significantly extended progression-free survival (PFS) in those patients (P. G. Richardson et al., Panorama 1: A randomized, double-blind, phase 3 study of panobinostat or placebo plus bortezomib and dexamethasone in relapsed or relapsed and refractory multiple myeloma, J Clin Onc. 32:5s, 2014 (suppl; abstr 8510). Although this clinical trial showed that panobinostat increased PFS, there were serious toxicities observed in some patients. In the PANORAMA clinical study there were severe and fatal ischemic events, severe arrhythmias and ECG changes in patients receiving panobinostat. Accordingly there is a need to reduce toxicities in patients receiving panobinostat in combination with receiving at least one other drug.

### SUMMARY OF THE INVENTION

The present disclosure provides for combinations and dosages of panobinostat for multiple myeloma that are improved with respect to safety, patient selectivity, in response to adverse events and drug-drug interactions. The claimed invention results in improved safety and outcomes for patients. More patients are able to use panobinostat, particularly in combination with another agent, for the treatment of multiple myeloma and thereby increasing patients' chances at completing their dosage cycles and receiving clinical benefits such as a longer time of progression free disease state.

### SUMMARY OF THE DRAWINGS

FIG. 1 Kaplan-Meier plot of progression-free survival PFS) in patients with multiple myeloma who received prior treatment with both bortezomib and an immunomodulatory agent.

### DESCRIPTION OF THE INVENTION

The term “treating” or “treatment” as used herein comprises a treatment relieving, reducing or alleviating at least one symptom in a subject, increasing progression-free survival, overall survival, extending duration of response or effecting a delay of progression of a disease. For example, treatment can be the diminishment of one or several symptoms of a disorder or complete eradication of a disorder, such as cancer. Within the meaning of the present disclosure, the term “treatment” also denotes to arrest, delay the onset (i.e., the period prior to clinical manifestation of a disease) and/or reduce the risk of developing or worsening a disease in a patient, e.g., a mammal or human. The term “prevent”, “preventing” or “prevention” as used herein comprises the prevention of at least one symptom associated with or caused by the state, disease or disorder being prevented.

The term “patient” as used herein is a human suffering from cancer, especially multiple myeloma.

The term “comprising” is used herein in its open-ended and non-limiting sense unless otherwise noted. In a more limited embodiment, “comprising” can be replaced by “consisting of”, which is no longer open-ended. In a most limited version it can include only features, steps or values as listed in the respective embodiment.

The terms “a” and “an” and “the” and similar references in the context of describing the disclosure (especially in the context of the following claims) are to be construed to cover both the singular and the plural, unless otherwise indicated herein or clearly contradicted by context. Where the plural form is used for compounds, patients, cancers and the like, this is taken to mean also a single compound, patient, or the like.

The term “pharmaceutically effective amount” or “clinically effective amount” of a therapeutic agent is an amount sufficient to provide an observable improvement over the baseline clinically observable signs and symptoms of the disorder treated with the therapeutic agent.

The term about” or “approximately” shall have the meaning of within 10%, more preferably within 5%, of a given value or a range.

As used herein, the term “carrier” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. The pharmaceutical composition can be subjected to conventional pharmaceutical operations and/or can contain conventional inert diluents, lubricating agents, as well as adjuvants, such as wetting agents, etc.

The term “assaying” is used to refer to the act of identifying, screening, probing or determining, which act may be performed by any conventional means. For example, a sample may be assayed for the presence of a particular marker by using an ELISA assay, a Northern blot, imaging, etc. to detect whether that marker is present in the sample. The terms “assaying” and “determining” contemplate a transformation of matter, e.g., a transformation of a biological sample, e.g., a blood sample or other tissue sample, from one state to another by means of subjecting that sample to physical testing. Further, as used herein, the terms “assaying” and “determining” are used to mean testing and/or measuring. The phrase “assaying a biological sample from the patient for . . . ” and the like is used to mean that a sample may be tested (either directly or indirectly) for either the presence or absence of a given factor or for the level of a particular factor. It will be understood that, in a situation where the presence of a substance denotes one probability and the absence of a substance denotes a different probability, then either the presence or the absence of such substance may be used to guide a therapeutic decision.

The phrase “receiving data” is used to mean obtaining possession of information by any available means, e.g., orally, electronically (e.g., by electronic mail, encoded on diskette or other media), written, etc.

As used herein, “selecting” and “selected” in reference to a patient is used to mean that a particular patient is specifically chosen from a larger group of patients on the basis of (due to) the particular patient having a predetermined set of criteria. Similarly, “selectively treating” refers to providing treatment to a patient having a particular disease, where that patient is specifically chosen from a larger group of patients on the basis of the particular patient having predetermined criteria. Similarly, “selectively administering” refers to administering a drug to a patient that is specifically chosen from a larger group of patients on the basis of (due to) the particular patient having predetermined criteria. By selecting, selectively treating and selectively administering, it is meant that a patient is delivered a personalized therapy based on the patient's particular biology, rather than being delivered a standard treatment regimen based solely on the patient having a particular disease. Selecting, in reference to a method of treatment as used herein, does not refer to fortuitous treatment of a patient that has the biomarker, but rather refers to the deliberate choice to administer treatment to a patient based on the patient having the biomarker. Thus, selective treatment differs from standard treatment, which delivers a particular drug to all patients, regardless of their biomarker.

As used herein, “predicting” indicates that the methods described herein provide information to enable a health care provider to determine the likelihood that an individual having the disorder will respond to or will respond more favorably to treatment. It does not refer to the ability to predict response with 100% accuracy. Instead, the skilled artisan will understand that it refers to an increased probability, e.g. of response.

As used herein, “likelihood” and “likely” is a measurement of how probable an event is to occur. It may be used interchangeably with “probability”. Likelihood refers to a probability that is more than speculation, but less than certainty. Thus, an event is likely if a reasonable person using common sense, training or experience concludes that, given the circumstances, an event is probable. In some embodiments, once likelihood has been ascertained, the patient may be treated (or treatment continued, or treatment proceed with a dosage increase) with the test compound. In one embodiment, the “likelihood” and “likely” denote a chance in percent of how probable an event is to occur.

The phrase “increased likelihood” refers to an increase in the probability that an event will occur. For example, some methods herein allow prediction of whether a patient will display an increased likelihood of responding to treatment with the test molecule or an increased likelihood of responding better to treatment with the test molecule. In one embodiment the increased likelihood means that there is more than 50% chance, more than 60% chance, more than 70% or more than 80% chance that an event will occur. Equally, a decreased likelihood means, that the chance is lower than 50%, lower than 60%, lower than 70% or lower than 80%, respectively, that an event will occur.

The term “a combined preparation”, as used herein defines especially a “kit of parts” in the sense that the active ingredients as defined above can be dosed independently or by use of different fixed combinations with distinguished amounts of the ingredients, i.e., simultaneously or at different time points. The parts of the kit can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the active ingredients. The ratio of the total amounts of the active ingredient 1 to the active ingredient 2 to be administered in the combined preparation can be varied, e.g., in order to cope with the needs of a patient sub-population to be treated or the needs of the single patient which different needs can be due to age, sex, body weight, etc. of the patients. Preferably, there is at least one beneficial effect, e.g., a mutual enhancing of the effect of the first and second active ingredient, in particular a synergism, e.g. a more than additive effect, additional advantageous effects, less side effects, a combined therapeutical effect in a non-effective dosage of one or both of the first and second active ingredient, and especially a strong synergism the first and second active ingredient.

The compounds described above are often used in the form of a pharmaceutically acceptable salt. Pharmaceutically acceptable salts include, when appropriate, pharmaceutically acceptable base addition salts and acid addition salts, for example, metal salts, such as alkali and alkaline earth metal salts, ammonium salts, organic amine addition salts and amino acid addition salts and sulfonate salts. Acid addition salts include inorganic acid addition salts, such as hydrochloride, sulfate and phosphate; and organic acid addition salts, such as alkyl sulfonate, arylsulfonate, acetate, maleate, fumarate, tartrate, citrate and lactate. Examples of metal salts are alkali metal salts, such as lithium salt, sodium salt and potassium salt; alkaline earth metal salts, such as magnesium salt and calcium salt, aluminum salt and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts. A preferred salt of panobinostat is the lactate salt, especially the anhydrous lactate form, described, e.g. in WO2007/146715.

Common Terminology Criteria for Adverse Events (CTCAE) is widely accepted throughout the oncology research community as the standard grading scale for adverse events. CTCAE is promulgated by the United States National Cancer Institute.

In one embodiment, the pharmaceutical compositions are gelatin capsules containing 20, 15 or 10 mg of panobinostat by weight of free base and the following inactive ingredients: magnesium stearate, mannitol, microcrystalline cellulose and pregelatinized starch. The capsules contain gelatin, FD&C Blue 1 (10 mg capsules), yellow iron oxide (10 mg and 15 mg capsules), red iron oxide (15 mg and 20 mg capsules) and titanium dioxide. The pharmaceutical composition can be used in the methods of the present disclosure.

The following Examples illustrate the disclosure described above; they are not, however, intended to limit the scope of the disclosure in any way. The beneficial effects of the panobinostat for use in the treatment according to the present disclosure, or methods as disclosed herein can also be determined by other test models known as such to the person skilled in the pertinent art. The aspects, advantageous features and preferred embodiments of the present invention summarized in the following items, respectively alone or in combination, further contribute improved administration of panobinostat.

### Dosage for Treatment of Multiple Myeloma

A starting dose of panobinostat can be 20 mg, taken orally once every other day for 3 doses per week in weeks 1 and 2 of each 21-Day cycle (week 3 being a rest cycle) for up to 8 cycles and continuing treatment for an additional 8 cycles for patients with clinical benefit who do not experience unresolved severe or medically significant toxicity. The total duration of treatment may be up to 16 cycles (48 weeks). That is in 21-Day Cycles 1 to 8 panobinostat can be administered on days 1, 3 and 5 (week 1) and days 8, 10, and 12 (week 2) and not administered on week 3, which is the rest period. In 21-Day Cycles 9 to 16 panobinostat can be administered on days 1, 3 and 5 (week 1) and days 8, 10, and 12 (week 2) and not administered on week 3, which is the rest period. For treating multiple myeloma panobinostat can be administered in combination with bortezomib and dexamethasone.

By way of example, bortezomib can be dosed at 1.3 mg/m2 given as an injection for 2 doses per week on the first up to 8 21-Day Cycles and 1 dose per week on the continuing up to 16 cycles. That is a 21-day cycle (week 3 being a rest cycle) at 2 doses per week for up to 8 cycles and continuing treatment for an additional 8 cycles (week 3 being a rest cycle) at 1 dose per week for patients with clinical benefit who do not experience unresolved severe or medically significant toxicity. By way of example bortezomib can be given in 21-Day Cycles 1 to 8 on days 1 and 4 (week 1) and days 8 and 11 (week 2) and not administered on week 3, which is the rest period. In 21-Day Cycles 9 to 16 bortezomib can be given on days 1 (week 1) and 8 (week 2) and would not be given during the rest period (week 3). The injection for bortezomib can be intravenous or subcutaneous. A subcutaneous injection may improve the safety of the administered combination with panobinostat without a reduction in efficacy.

Dexamethasone can be taken orally per scheduled day, preferably on a full stomach. The dosage of dexamethasone can be 20 mg. Dexamethasone can be dosed at 4 doses per week on the first up to 8 21-Day Cycles and 2 dose per week on the continuing up to 16 cycles. That is a 21-day cycle (week 3 being a rest cycle) at four doses per week for up to 8 cycles and continuing treatment for an additional 8 cycles (week 3 being a rest cycle) at 2 doses per week for patients with clinical benefit who do not experience unresolved severe or medically significant toxicity. By way of example dexamethonse can be given in 21-Day Cycles 1 to 8 on days 1, 2, 4 and 5 (week 1) and days 8, 9, 11 and 12 (week 2) and not administered on week 3, which is the rest period. In 21-Day Cycles 9 to 16 dexamethasone can be given on days 1 and 2 (week 1) and 8 and 9 (week 2) and would not be given during the rest period (week 3).

The administration of panobinostat in combination with a proteasome inhibitor such as bortezomib can be of benefit to patients that have become resistant or refractory to bortezomib or an immunomodulatory drug (IMiD), such as thalidomide, lenalidomide or pomalidomide. Additional prior treatments or lines of therapies can include chemotherapeutic agents such as corticosteroids (e.g. dexamethasone), melphalan or cyclophosphamide. Panobinostat can be administered to the patient after the patient has become resistant to one or more prior therapies with bortezomib, and an IMiD or both. For example, panobinostat can be administered to multiple myeloma patients who receive at least two prior lines of therapies, including bortezomib and an IMiD.

The present invention therefore provides dosage regimens for patients suffering from multiple myeloma wherein the multiple myeloma has become resistant or refractory to bortezomib or an immunomodulatory drug (IMiD), such as thalidomide, lenalidomide or pomalidomide. Also provided herein are dosage regimens for patients suffering from multiple myeloma who receive additional or have received additional prior therapies as disclosed herein.

### Patient Screening and Monitoring CBC, ECG and Electrolytes

Prior to the start of panobinostat treatment a patient is optionally screened for Complete Blood Count (CBC) before initiating treatment. The baseline platelet count is verified to be at least 100×109/L and the baseline absolute neutrophil count (ANC) is verified to be at least 1.5×109/L. If values are below these numbers the patient is not given panobinostat treatment. The CBC is monitored at least weekly during treatment.

Prior to the start of panobinostat treatment a patient is optionally screened by performing an electrocardiogram (ECG) prior to the start of therapy. The QTcF (corrected QT interval using Fridericia's formula,) is verified to less than <480 msec prior to initiation of treatment with panobinostat. If the value is below this number, the patient is not given panobinostat treatment. QTcF is monitored during treatment. If during treatment with panobinostat, the QTcF increases to ≧480 msec, treatment is interrupted. Any electrolyte abnormalities are corrected. By way of example, ECGs can be performed at baseline and prior to initiation of each cycle for the first 8 cycles.

Panobinostat may prolong cardiac ventricular repolarization (QT interval). In the randomized multiple myeloma trial, QTc (corrected QT using a standard computer-based ECG machine) prolongation with values between 451 ms to 480 ms occurred in 10.8% of panobinostat treated patients. Events with values of 481 ms to 500 ms occurred in 1.3% of panobinostat treated patients. A maximum QTcF increase from baseline of between 31 msec and 60 msec was reported in 14.5% of panobinostat treated patients. A maximum QTcF increase from baseline of >60 ms was reported in 0.8% of panobinostat treated patients. No episodes of QTcF prolongation >500 msec have been reported with the dose of 20 mg panobinostat in the randomized multiple myeloma trial conducted in combination with bortezomib and dexamethasone. Pooled clinical data from over 500 patients treated with single agent panobinostat in multiple indications and at different dose levels have shown that the incidence of CTC Grade 3 QTc prolongation (QTcF>500 msec) was approximately 1% overall and 5% or more at a dose of 60 mg or higher.

Optionally, testing of serum electrolytes, including potassium and magnesium, can be done at baseline and abnormal electrolyte values can be corrected before treatment. Monitoring of serum electrolytes can be done throughout therapy. Monitoring can be conducted prior to the start of each cycle and at day 11 of cycles 1-8 and at the start of each cycle for cycles 9- to 16.

### Patient Screening and Monitoring Hepatic Impairment

Surprisingly it has been found that patients with some degree of hepatic impairment can be administered a combination of panobinostat and bortezomib. For example, a starting dose of 20 mg of panobinostat can be reduced to 15 mg in patients with mild hepatic impairment and 10 mg in patients with moderate hepatic impairment. Preferably panobinostat is not used in patients with severe hepatic impairment. After starting patients on a dose of panobinostat, they are preferably monitored frequently for adverse events and the dose adjusted as needed for toxicity. Frequency of monitoring patients can vary. For example patients can be monitored once a week, twice a week or every day they are receiving panobinostat or one of its combination partners. Mild hepatic impairment is bilirubin≦1×the upper limit of the normal range (“ULN”) and aspartate aminotransferase (“AST”) >1×ULN, or bilirubin>1.0-1.5×ULN and any amount of AST above ULN is present). Moderate hepatic impairment is bilirubin>1.5×-3.0×ULN and any amount of AST above ULN is present. Severe hepatic is bilirubin≧3.0×ULN and any amount of AST above ULN is present.

In a pharmacokinetic trial, patients with mild (bilirubin≦1×ULN and AST>1×ULN, or bilirubin>1.0-1.5×ULN and any AST) or moderate (bilirubin>1.5×-3.0×ULN, any AST) hepatic impairment (NCI-ODWG criteria) had increased AUC of panobinostat by 43% and 105%, respectively. The starting dose of panobinostat in patients with mild or moderate hepatic impairment is reduced. Use in patients with severe hepatic impairment is avoided. Patients with hepatic impairment are monitored frequently for adverse events.

Alternatively when moderate hepatic impairment is shown in patient screening, the dosage of panobinostat can be reduced to 10 mg in the first cycle and for the subsequent cycles the dosage can optionally be escalated up to 15 mg based on patient tolerability. Optionally and in addition, the starting dosage of bortezomib for patients with moderate hepatic impairment may be reduced to a bortezomib dose to 0.7 mg/m² in the first treatment cycle. Dose escalation for bortezomib can be increased to 1.0 mg/m² or further dose reduction to 0.5 mg/m² in subsequent cycles based on patient tolerability.

The effect of hepatic impairment on the pharmacokinetics of panobinostat was evaluated in a phase 1 study in 24 patients with advanced cancer with varying degrees of hepatic impairment. In patients with NCI-CTEP class mild (i.e., Group B) and moderate (i.e., Group C) hepatic impairment, AUC_{0-inf} increased 43% and 105% compared to the group with normal hepatic function, respectively. The relative change in Cₘₐₓ followed a similar pattern. The effect of severe hepatic impairment was indeterminate in this study due to the small sample size (n=1). A dose modification is recommended for patients with mild and moderate hepatic impairment.

### Dosage Adjustments Due to Toxicity

Dose and/or schedule modification of panobinostat may be required based on toxicity. Management of adverse drug reactions may require treatment interruption and/or dose reductions. If dose reduction is required, the dose of panobinostat can be reduced in increments of 5 mg (i.e., from 20 mg to 15 mg, or from 15 mg to 10 mg). Panobinostat is discontinued rather than reducing the dosing of panobinostat to below 10 mg given 3 times per week. The same treatment cycles (e.g. a three 3-week treatment cycle) is kept when reducing dose. The following Tables 1-5 also list bortezomib (BTZ) dose modifications that can be made according to the toxicity related adverse events found in patients.

TABLE 1
Thrombocytopenia

| Platelets <50 × | Platelets <50 × | Platelets <25 × |
|---|---|---|
| 10⁹/L CTCAE | 10⁹/L with bleeding | 10⁹/L CTCAE |
| grade 3 | CTCAE grade 3 | grade 4 |
| Maintain | Interrupt | Interrupt |
| panobinostat dose | panobinostat. | panobinostat. |
| Monitor platelet | Monitor platelet | Monitor platelet |
| counts at least | counts at least | counts at least |
| weekly | weekly until ≧50 × | weekly until ≧50 × |
| | 10⁹/L, then | 10⁹/L, then |
| | restart at reduced | restart at reduced |
| | dose | dose |

| | |
|---|---|
| Maintain BTZ | Interrupt bortezomib until thrombocytopenia |
| dose | resolves to ≧75 × 10⁹/L |
| | if only one dose was omitted prior to correction |
| | to these levels, restart bortezomib at same dose |
| | if 2 or more doses were omitted consecutively, |
| | or within the same cycle, bortezomib should be |
| | restarted at a reduced dose |

Thrombocytopenia is a low blood platelet count. A decrease in the number of platelets to less than 50.0 × 10e9/L is CTCAE grade 3 and less than 25.0 × 10e9/L is CTCAE grade 4.

TABLE 2
Neutropenia

| | ANC 0.5 to | | |
|---|---|---|---|
| ANC 0.75 | 0.75 × 10⁹/L | ANC <1.0 × 10⁹/L | |
| to 1.0 × | CTCAE | (CTCAE grade 3) | |
| 10⁹/L | grade 3 | with febrile | ANC <0.5 × |
| CTCAE | (2 or more | Neutropenia (any | 10⁹/L CTCAE |
| grade 3 | occurrences) | grade) | grade 4 |
| Maintain | Interrupt | Interrupt | Interrupt |
| panobinostat | panobinostat | panobinostat until | panobinostat until |
| dose | until ANC ≧1.0 × | febrile neutropenia | ANC ≧1.0 × |
| | 10⁹/L | resolves and | 10⁹/L, then |
| | and restart at | ANC ≧1.0 × 10⁹/ | restart at reduced |
| | same dose | L, then restart | dose |
| | | at reduced dose | |

| | |
|---|---|
| Maintain | Interrupt bortezomib until febrile |
| bortezomib dose | neutropenia resolves and ANC >1.0 × |
| | 10⁹/L |
| | if only one dose was omitted prior to |
| | correction to these levels, restart |
| | bortezomib at same dose, |
| | if two or more doses were omitted |
| | consecutively, or within the same |
| | cycle, bortezomib should be restarted |
| | at a reduced dose |

Absolute neutrophil count (ANC) is a measure of the number of neutrophil (also known as neutrophil granulocytes) present in the blood. ANC is a well know measure in the art and is given by the equation ANC = (% neutrophils + % bands) × (WBC)/(100). WBC is white blood cells per microliter of blood. The unit ANC is per microliter of blood.

TABLE 3

| | |
|---|---|
| Anemia | Hemoglobin < 8 g/dL |

Interrupt panobinostat until hemoglobin ≧ 10 g/dL
Restart at reduced dose
Anemia is a lower than normal amount of red blood cells and can be diagnosed by measuring in a sample of blood the amount of hemoglobin (Hb). Normal levels of hemoglobin are Hb 12-16 g/dL (women) or Hb 13.5-17.5 g/dL (men). Mild anemia is Hb 10-12 g/dL (women) or Hb 10-13.5 g/dL (men). Moderate anemia is Hb 8-<10 g/dL and severe anemia is Hb <8 g/dL.

TABLE 4
Diarrhea

| | Severe Diarrhea | |
|---|---|---|
| | (≧7 stools/day), | |
| Moderate Diarrhea | intravenous (IV) | Life-threatening |
| 4 to 6 stools/day | fluids or hospitalization | Diarrhea |
| CTCAE grade 2 | required CTCAE grade 3 | CTCAE grade 4 |
| Interrupt panobinostat | Interrupt | Permanently |
| until resolved | panobinostat until | discontinue |
| Restart at same dose | resolved. | panobinostat |
| | Restart at reduced | |
| | dose level | |
| Consider Interruption of | Interrupt bortezomib | Permanently |
| bortezomib until resolved | until resolved | discontinue |
| Restart at same dose | Restart at reduced | bortezomib |
| | dose level | |

Diarrhea is a disorder characterized by frequent and watery bowel movements. CTCAE grade 1 is less than an increase of 4 stools a day over baseline and/or a mild increase of ostomy output over baseline. CTCAE grade 2 is an increase of 4 to 6 stools a day over baseline and/or a moderate increase of ostomy output over baseline. CTCAE grade 3 is at least 7 stools a day over baseline, incontinence, hospitalization indicated, or a severe increase of ostomy output over baseline, or a limited ability to perform self care.

TABLE 5
Nausea or Vomiting

| | Severe/Life-threatening |
|---|---|
| Severe Nausea | Vomiting |
| CTCAE grade 3/4 | CTCAE grade 3/4 |
| Interrupt panobinostat until | Interrupt panobinostat until |
| resolved, then restart at | resolved, then restart at |
| reduced dose | reduced dose |

### Myelosuppression

The dose of panobinostat in patients who have thrombocytopenia, neutropenia or anemia is interrupted or reduced according to instructions in the corresponding Tables 1-3. For patients with severe thrombocytopenia, platelet transfusions are considered. Panobinostat treatment is discontinued if thrombocytopenia does not improve despite the recommended treatment modifications or if repeated platelet transfusions are required.

In the event of Grade 3 or 4 neutropenia, dose reduction and/or the use of growth factors (e.g., G-CSF) are considered. Panobinostat is discontinued if neutropenia does not improve despite dose modifications, colony-stimulating factors, or in case of severe infection.

### Gastrointestinal Toxicity

Gastrointestinal toxicity is common in patients treated with panobinostat. Patients who experience diarrhea, nausea, or vomiting may require treatment interruption or dose reduction, see corresponding tables. At the first sign of abdominal cramping, loose stools, or onset of diarrhea, patients should be treated with anti-diarrheal medication (e.g., loperamide). Consider and administer prophylactic anti-emetics as clinically indicated.

### Other Adverse Drug Reactions

For patients experiencing Grade 3/4 adverse drug reactions other than thrombocytopenia, neutropenia, or gastrointestinal toxicity, the recommendation is the following. CTCAE (Common Terminology Criteria for Adverse Events) Grade 2 toxicity recurrence and CTCAE Grade 3 and 4—the dose is omitted until recovery to CTCAE Grade 1 or less and treatment restarted at a reduced dose. CTCAE Grade 3 or 4 toxicity recurrence, a further dose reduction may be considered once the adverse events have resolved to CTCAE Grade 1 or less.

### CYP3A Inhibitors

The starting dose of panobinostat is reduced to 10 mg when co-administered with strong CYP3A inhibitors (e.g., boceprevir, clarithromycin, conivaptan, indinavir, itraconazole, ketoconazole, lopinavir/ritonavir). Strong, moderate, or weak CYP3A inhibitors are defined as those drugs that increase the AUC of oral midazolam or other CYP3A substrates ≧5-fold, 2-5-fold, and 1.25-2-fold, respectively.

### CYP3A Inducers

The effect of hepatic impairment on the pharmacokinetics of panobinostat was evaluated in a phase 1 study in 24 patients with advanced cancer with varying degrees of hepatic impairment. In patients with NCI-CTEP class mild (i.e., Group B) and moderate (i.e., Group C) hepatic impairment, AUC_{0-inf} increased 43% and 105% compared to the group with normal hepatic function, respectively. The relative change in Cₘₐₓ followed a similar pattern. The effect of severe hepatic impairment was indeterminate in this study due to the small sample size (n=1). A dose modification is recommended for patients with mild and moderate hepatic impairment.

### CYP3DG Substrate

Coadministration of a single 60 mg dextromethorphan (DM) dose with panobinostat (20 mg once per day, on Days 3, 5, and 8) increased the Cmax and AUC_{0-∞} of DM by 20% to 200% and 20% to 130% (interquartile ranges), respectively, compared to when DM was given alone in 14 patients with advanced cancer. These DM exposures were extremely variable (CV %>150%). Coadministration of panobinostat is to be avoided with sensitive CYP2D6 substrates or CYP2D6 substrates that have a narrow therapeutic index, which is a difference of twofold or less between the median effective dose (ED50) and the median toxic dose (TD50).

### Infections

Panobinostat treatment should not be initiated in patients with active infections. Patients are monitored for signs and symptoms of infections during treatment; if a diagnosis of infection is made, appropriate anti-infective treatment is instituted promptly and interruption or discontinuation of panobinostat is considered.

### Clinical Trial

The efficacy and safety of panobinostat in combination with bortezomib and dexamethasone was evaluated in a randomized, double-blind, placebo-controlled, multicenter study in patients with relapsed multiple myeloma who had received 1 to 3 prior lines of therapy.

Patients received bortezomib (1.3 mg/m2 injected intravenously) with dexamethasone (20 mg) in addition to panobinostat 20 mg (or placebo), taken orally every other day, for 3 doses per week in Weeks 1 and 2 of each 21-day cycle. Treatment was administered for a maximum of 16 cycles (48 weeks).

A total of 768 patients were randomized in a 1:1 ratio to receive either the combination of panobinostat, bortezomib, dexamethasone (n=387) or placebo, bortezomib, dexamethasone (n=381), stratified by prior use of bortezomib and the number of prior lines of anti-myeloma therapy. Demographics and baseline disease characteristics were balanced between arms. The median age was 63 years (range 28 to 84); 42% of patients were older than 65 years; 53% of patients were male. The ECOG (Eastern Cooperative Oncology Group) performance status was 0 to 1 in 93% of patients. The median number of prior therapies was 1; 48% of patients received 2 or 3 prior lines of therapy. More than half (57%) of the patients had prior stem cell transplantation. The most common prior antineoplastic therapies were corticosteroids (90%), melphalan (80%), thalidomide (53%), cyclophosphamide (47%), bortezomib (44%), and lenalidomide (19%). The median duration of follow-up was 29 months in both arms.

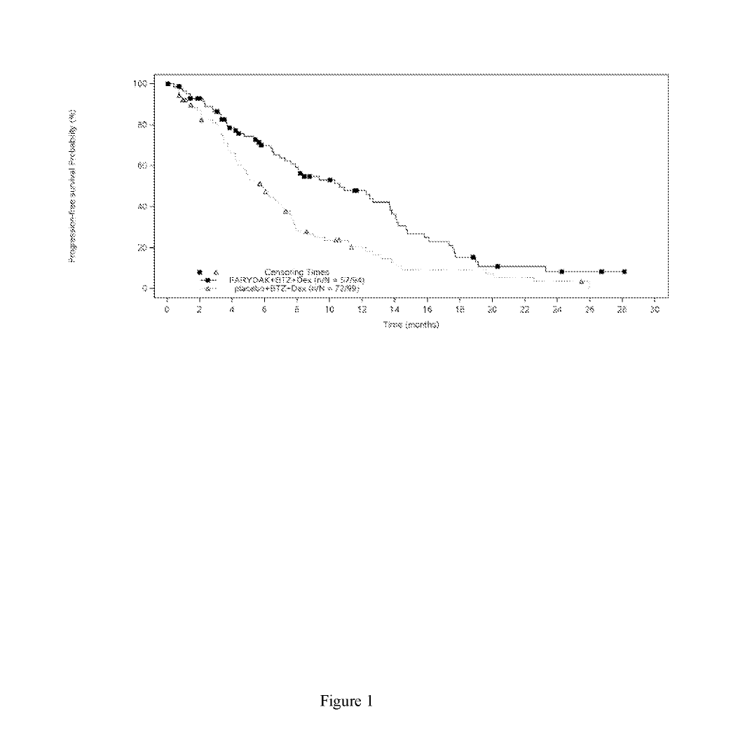

The primary endpoint was progression-free survival (PFS), using modified European Bone Marrow Transplant Group (EBMT) criteria, as assessed by the investigators. In the overall trial population, the median PFS (95% CI) was 12 months (10.3, 12.9) in the panobinostat, bortezomib, dexamethasone arm and 8.1 months (7.6, 9.2) in the placebo, bortezomib, dexamethasone arm, [HR: 0.63 (95% CI: 0.52, 0.76)]. At the time of interim analysis, overall survival was not statistically different between arms. The efficacy and safety in a subgroup analysis of 193 patients who had received prior treatment with both bortezomib and an immunomodulatory agent and a median of 2 prior therapies as the benefit:risk appeared to be greater in this more heavily pretreated population than in the overall trial population. Of these 193 patients, 76% of them had received ≧2 prior lines of therapy. The median PFS (95% CI) was 10.6 months (7.6,13.8) in the panobinostat, bortezomib, and dexamethasone arm and 5.8 months (4.4, 7.1) in the placebo, bortezomib, and dexamethasone arm [HR: 0.52 (0.36, 0.76]. Efficacy results are summarized in Table 1 and the Kaplan-Meier curves for PFS are provided in FIG. 1 (PAN=panobinostat, BTZ=bortezomib and DEX=dexamethasone).

TABLE 1
Number of patients at risk

| | Months | | | | | | | |
|---|---|---|---|---|---|---|---|---|
| | 0 | 2 | 4 | 6 | 8 | 10 | 12 | 14 |
| PAN + BTZ + DEX | 94 | 74 | 57 | 46 | 39 | 32 | 25 | 19 |
| placebo + BTZ + DEX | 99 | 72 | 53 | 37 | 20 | 16 | 11 | 7 |
| | Months | | | | | | | |
| | 16 | 18 | 20 | 22 | 24 | 26 | 28 | 30 |
| PAN + BTZ + DEX | 13 | 8 | 5 | 4 | 3 | 2 | 1 | 0 |
| placebo + BTZ + DEX | 5 | 5 | 4 | 3 | 2 | 0 | 0 | 0 |

1. A combination comprising panobinostat, or a pharmaceutically acceptable salt thereof, and bortezomib for use in a method of treatment of multiple myeloma in a patient, where in the method comprises;
assaying a blood sample from the patient;
determining if the patient has no hepatic impairment or has mild, moderate or severe hepatic impairment; and
administering to the patient:
a starting dosage of 20 mg panobinostat if the patient has no or mild hepatic impairment;
a starting dosage of 10 mg of panobinostat if the patient has moderate hepatic impairment; and
not administering panobinostat if the patient has severe hepatic impairment;
wherein mild hepatic impairment is bilirubin≦1×the upper limit of the normal range (“ULN”) and aspartate aminotransferase (“AST”) >1×ULN, or bilirubin>1.0-≦1.5×ULN and any amount of AST above ULN is present;
wherein moderate hepatic impairment is bilirubin>1.5×-≦3.0×ULN and any amount of AST above ULN is present;
wherein severe hepatic impairment is bilirubin>3.0×ULN and any amount of AST above ULN is present; and
wherein panobinostat, or a pharmaceutically acceptable salt thereof (e.g. the lactate or the anhydrous lactate salt thereof) is in the form of an oral dosage form.

2. A combination according to claim 1 for use according to claim 1, wherein the method further comprises administering to the patient an effective dosage of dexamethasone.

3. A combination according to claim 2, wherein the multiple myeloma is resistant or refractory to prior treatments.

4. A combination according to claim 3, wherein the dosage of bortezomib is 1.3 mg/m² administered as an injection.

5. A combination according to claim 3, wherein the bortezomib is administered at a dosage of 0.7 mg/m² and wherein the patient has mild hepatic impairment.

6. A combination according to claim 5, wherein the prior treatments have been treatment with bortezomib or an immunomodulatory agent.

7. A combination according to claim 6, wherein the prior treatments have included both bortezomib and an immunomodulatory agent.

8. A combination according to claim 6, wherein the prior treatments were a chemotherapeutic agent.

9. A method of treating a human patient having multiple myeloma comprising:
assaying a blood sample from the patient;
determining if the patient has no hepatic impairment or has mild, moderate or severe hepatic impairment; and
administering to the patient:
a starting dosage of 20 mg of panobinostat if the patient has no hepatic impairment;
a starting dosage of 15 mg of panobinostat if the patient has mild hepatic impairment;
a starting dosage of 10 mg of panobinostat if the patient has moderate hepatic impairment; and
not administering panobinostat if the patient has severe hepatic impairment
wherein mild hepatic impairment is bilirubin≦1×the upper limit of the normal range (“ULN”) and aspartate aminotransferase (“AST”) >1×ULN, or bilirubin>1.0 to 1.5×ULN and any amount of AST above ULN is present);
wherein moderate hepatic impairment is bilirubin>1.5× to 3.0×ULN and any amount of AST above ULN is present;
wherein severe hepatic is bilirubin>3.0×ULN and any amount of AST above ULN is present; and
wherein the term panobinostat is the molecule itself or a pharmaceutically acceptable salt thereof.

10. The method of claim 6, wherein the patient is also administered an effective dosage of dexamethasone.

11. The method of claim 6, wherein the multiple myeloma is resistant or refractory to prior treatments.

12. The method of claim 9, wherein the prior treatments have been treatment with bortezomib or an immunomodulatory agent.

13. The method of claim 12, wherein the prior treatments have included both bortezomib and an immunomodulatory agent.

14. The method of claim 9, wherein the prior treatments included or consisted of treatment with a chemotherapeutic agent.
